(19) United States
(12) Patent Application Publication
(10) Pub. No.: US 2025/0124065 A1
(43) Pub. Date: Apr. 17, 2025

(54) INTERACTIVE EXPERT INFORMATION PLATFORM AND METHOD

(71) Applicant: Mediflix LLC, New York, NY (US)

(72) Inventors: Steve Leber, New York, NY (US); Sanjay Patel, Portland, ME (US)

(21) Appl. No.: 18/917,629

(22) Filed: Oct. 16, 2024

Related U.S. Application Data
(60) Provisional application No. 63/544,358, filed on Oct. 16, 2023.

(51) Int. Cl. G06F 16/332; G06F 16/33; G06F 16/338; G06F 16/35; G16H 80/00

(52) U.S. Cl. CPC G06F 16/3329; G06F 16/3344; G06F 16/338; G06F 16/353; G16H 80/00

(57) ABSTRACT

A medical information system and method comprise at least one processor coupled to at least one computer storage device; a network interface enabling connectivity with one or more medical information systems and with one or more user devices; an artificial intelligence (AI) engine configured to process a request from a user device to interpret a context and a meaning from the request and to generate a response based on context and meaning of the request and data and information from the one or more medical information systems; and a user interface module configured to output the response. The AI engine can be configured to generate an interactive audiovisual response, which can include content search and generation.

### CROSS REFERENCE(S) TO RELATED APPLICATIONS

The present application claims priority under 35 USC 119(e) to U.S. Provisional Patent Application No. 63/544,358, entitled INTERACTIVE MEDICAL INFORMATION PLATFORM, filed Oct. 16, 2023, the contents of which are incorporated herein by reference in its entirety.

### FIELD OF INTEREST

The present inventive concepts relate to the field of computer-based systems useful in connecting consumers with medical information, preferably with artificial intelligence and/or machine learning to provide a high quality, informative user experience.

### SUMMARY

In accordance with various aspects of the inventive concepts, provided is a medical information system, comprising: at least one processor coupled to at least one computer storage device; a network interface enabling connectivity with one or more medical information sources and with one or more user devices; an artificial intelligence (AI) engine. The AI engine is configured to: process a question from a user device to determine a context and a meaning from the question; generate search criteria from the context and the meaning and to search the one or more medical information sources to find expert content responsive to the question.

In various embodiments, the AI engine further comprises a classification module configured to determine a classification of the question from the context and the meaning.

In various embodiments, the classification module is configured to classify the question has belonging to a group consisting of: emergency, contact doctor, searchable, and unknown.

In various embodiments, the AI module is configured to search the one or more medical information sources when the classification is searchable.

In various embodiments, the expert content includes video content.

In various embodiments, the expert video content is prerecorded.

In various embodiments, the AI engine further comprises a tagging module configured to determine tags from the question.

In various embodiments, the tags are determined from a set of tags that include predefined, stored tags.

In various embodiments, the tagging module is configured to use machine learning to determine the tags.

In various embodiments, the search criteria include the tags and the AI module further includes a similarity search module configured to search the one or more medical information sources to determine content related to the question based, at least in part, of the tags.

In various embodiments, the similarity search module is configured to rank and/or order the determined content.

In various embodiments, the similarity search module is configured to determine the content as content that is semantically similar.

In various embodiments, the AI module further includes: an advice match rating module configured to assess the determined content to determine a best match for the question.

In various embodiments, the one or more medical information sources include vector databases and the search criteria includes at least one vector.

In various embodiments, the system further comprises a user interface module configured to format the expert content for display on a user device.

In various embodiments, the user interface module is further configured to format the expert content into different regions on the display.

In various embodiments, the different regions include panels or tiles, wherein a tile can be user-selectable content.

In various embodiments, each panel includes one or more tiles.

In various embodiments, each panel includes a specific type of content, including audio or video content.

In various embodiments, each panel includes content related to a different question.

In various embodiments, the user interface module is further configured to provide mechanisms for voice input and text input.

In various embodiments, the user interface module is further configured to include user-selected related questions in the display, each related question related to the user question.

In various embodiments, the user interface module is further configured to include user-selectable content from a plurality of different experts, including content related to the user question.

In various embodiments, the user interface module is further configured to include provide a chat session with the user.

In various embodiments, the chat session is with an expert related to the question.

In various embodiments, the user interface module is further configured to enable to the user to share, save, and/or the content.

In various embodiments, the user interface module is further configured to enable the user to enter a subsequent question.

In various embodiments, the user interface module is further configured to provide a graphical mechanism to enable the user to request personalized advice.

In various embodiments, the AI engine includes generative AI functionality.

In various embodiments, the AI engine is configured to generate at least some of the expert content.

In various embodiments, the AI engine is further configured to determine a language of the user and output the expert content in the language of the user.

In accordance with another aspect of the inventive concepts, provided is a computer-based method of providing medical information, comprising: providing at least one processor coupled to at least one computer storage device; providing a network interface enabling connectivity with one or more medical information sources and with one or more user devices; executing a computer program product comprising an artificial intelligence (AI) engine. The AI engine performs: processing a question from a user device to determine a context and a meaning from the question; generating search criteria from the context and the meaning and searching the one or more medical information sources to find expert content responsive to the question.

In various embodiments, the method further comprises determining a classification of the question from the context and the meaning.

In various embodiments, the method further comprises classifying the question has belonging to a group consisting of: emergency, contact doctor, searchable, and unknown.

In various embodiments, the method further comprises searching the one or more medical information sources when the classification is searchable.

In various embodiments, the expert content includes video content.

In various embodiments, the expert video content is prerecorded.

In various embodiments, the method further comprises determining tags from the question.

In various embodiments, the method further comprises determining the tags from a set of tags that include predefined, stored tags.

In various embodiments, the method further comprises using machine learning to determine the tags.

In various embodiments, the search criteria include the tags and the method further comprises: searching the one or more medical information sources to determine content related to the question based, at least in part, of the tags.

In various embodiments, the method further comprises ranking and/or ordering the determine content.

In various embodiments, the method further comprises determining the content as content that is semantically similar.

In various embodiments, the method further comprises assessing the determined content to determine a best match for the question.

In various embodiments, the one or more medical information sources include vector databases and the search criteria includes at least one vector.

In various embodiments, the method further comprises formatting the expert content for display on a user device.

In various embodiments, the method further comprises formatting the expert content into different regions on the display.

In various embodiments, the different regions include panels or tiles, wherein a tile can be user-selectable content.

In various embodiments, each panel includes one or more tiles.

In various embodiments, each panel includes a specific type of content, including audio or video content.

In various embodiments, each panel includes content related to a different question.

In various embodiments, the method further comprises providing mechanisms for voice input and text input.

In various embodiments, the method further comprises including user-selected related questions in the display, each related question related to the user question.

In various embodiments, the method further comprises including user-selectable content from a plurality of different experts, including content related to the user question.

In various embodiments, the method further comprises including providing a chat session with the user.

In various embodiments, the chat is with an expert related to the question.

In various embodiments, the method further comprises enabling the user to share, save, and/or the content.

In various embodiments, the method further comprises enabling the user to enter a subsequent question.

In various embodiments, the method further comprises providing a graphical mechanism to enable the user to request personalized advice.

In various embodiments, the AI engine includes generative AI functionality.

In various embodiments, the method further comprises the AI engine generating at least some of the expert content.

In various embodiments, the method further comprises the AI engine determining a language of the user and outputting the expert content in the language of the user.

### BRIEF DESCRIPTION OF THE DRAWINGS

The present invention will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the invention. In the drawings:

FIG. 1 is a functional block diagram showing an embodiment of a medical information system, in accordance with aspects of the inventive concepts;

FIGS. 2A through 2H depict embodiments of flow diagram implemented by the system of FIG. 1, in accordance with aspects of the inventive concepts;

FIGS. 3A through 3C show embodiments of flow diagrams of the AI system of FIG. 1; in accordance with aspects of the inventive concepts;

FIGS. 4A through 4M show an embodiment of screenshots resulting user's interaction with the system of FIG. 1 in accordance with aspects of the inventive concepts;

FIGS. 4N-4P shows embodiments of other guide screens presented with the classification module with does not determine the question to be searchable, in accordance with aspects of the inventive concepts;

FIGS. 5A through 5Q show another embodiment of screenshots resulting for a user's interaction with the system of FIG. 1, in accordance with aspects of the inventive concepts; and

FIGS. 6A through 6E show another embodiment of screenshots resulting from a user's interaction with the system of FIG. 1, in accordance with aspects of the inventive concepts.

### DETAILED DESCRIPTION OF PREFERRED EMBODIMENTS

Various aspects of the inventive concepts will be described more fully hereinafter with reference to the accompanying drawings, in which some exemplary embodiments are shown. The present inventive concept may, however, be embodied in many different forms and should not be construed as limited to the exemplary embodiments set forth herein.

It will be understood that, although the terms first, second, etc. are be used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

To the extent that functional features, operations, and/or steps are described herein, or otherwise understood to be included within various embodiments of the inventive concept, such functional features, operations, and/or steps can be embodied in functional blocks, units, modules, operations and/or methods. And to the extent that such functional blocks, units, modules, operations and/or methods include computer program code, such computer program code can be stored in a computer readable medium, e.g., such as non-transitory memory and media, that is executable by at least one computer processor.

An interactive expert information platform and method can be implanted in a variety contents and/or use cases. For the most part, the use case described herein is a medical expert use cases, e.g., wherein doctors are medical experts. But the inventive concepts could be implemented in any of a variety of contexts, particularly where expert advice is requested. Such other contents can include, but are not limited to, real estate, financial, legal, education, sport training (e.g. golf), and so forth.

In preferred embodiments, expert content provided by the system includes copyrighted work, and could also include non-copyrighted works. For example, the system can include and/or access databases of expert advice content from databases of indexed and copyrighted works. Such copyrighted works can be or include files including video, audio, graphic, and/or text.

A medical information system in accordance with the inventive concepts includes and utilizes artificial intelligence (AI) to enable and conduct interactive sessions with a user over a network. The network can be or include any now known of hereafter developed wired or wireless network, or combinations thereof. As examples, the network could include the Internet, World Wide Web, an intranet, a virtual private network and so forth. The user devices can include any of a variety of user interactive devices having a processor and user interface features. Such user devices can include, but are not limited to, a mobile phone, tablet, phablet, laptop, desktop computer, kiosk, watch, smart television, and so forth.

Within the context of medical information, users can be patients or caretakers in various embodiments. In various embodiments, medical professionals can also be users, either through providing content or consuming content, particularly in their fields. That is, the system can be used for selfcare and/or education, depending on the user.

The AI functionality can be embodied in an AI engine having general AI capabilities and generative AI capabilities. In some embodiments, the generative AI capabilities can create new content to support effective real-time interactions with a user. In some embodiments, the generative content can take the form of or include interactive video dialog between a user and the medical information utilizing the AI engine. In some embodiments, the AI engine can access a large video library containing medical information content from experts. The system can provide existing videos, AI generated videos, or combinations thereof. In some embodiments, the AI engine can be configured to alter an existing video in response to a request for information from a user, said alteration making the resulting video more responsive to the request. The AI engine can include bioinformatics functionality configured to process user information in the generation of responses to a user's request.

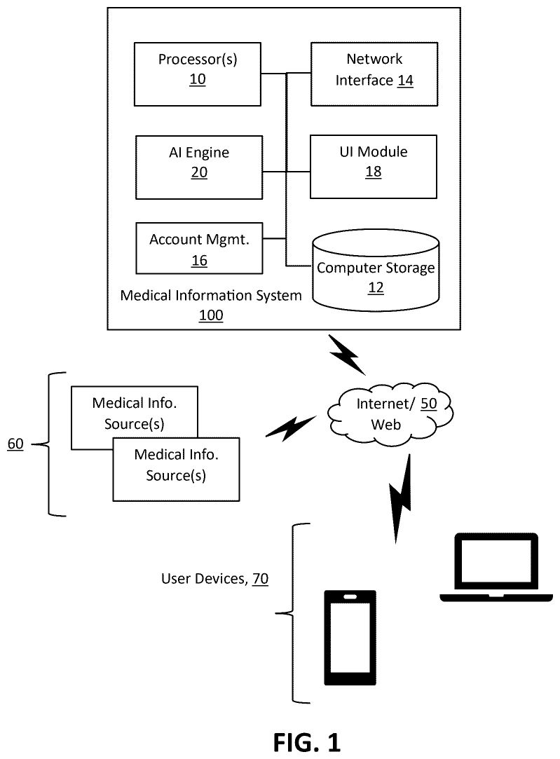

FIG. 1 is a functional block diagram showing an embodiment of a medical information system 100 in accordance with aspects of the inventive concepts. In this embodiment, a variety of function modules are shown as forming part of the system 100. In various embodiments, the functional modules may be collocated, distributed, or some combination thereof.

The system 100 can include one or more processors 10 coupled to one or more computer storage devices 12, which can be configured to store executable program code, data, user account information, and/or medical information content. The system 100 may be configured to communicate with external user devices 70 and medical information databases and/or sources 60, as well as other systems and data sources. Such medical information sources 60 can be used to provide real-time access to agents and/or medical professionals involved in real-time sessions with users seeking medical information, treatment, and/or advice. Additionally, or alternatively, such medical information sources 60 can include or provide access to libraries of expert videos categorized, related, and/or organized by condition, topic/subject, symptoms, provider, clinician, and/or patient.

In various embodiments, the medical information sources 60 can include diagnostic systems, treatment systems, provider systems, medical database systems, public health information resources, insurance provider systems, financial systems, educational systems and libraries, medical journal or publication resources, as well as other resources or systems that enable the medical information system 100 to effectively provide services to consumer and provider users.

The system 100 can include a network interface module 14 that enables communication with external systems, such as the user devices 70 and the medical information sources 60, and any other external system that may be useful for executing the functions of the system 100.

The system 100 can include an account management module 16 configured to establish and manage user accounts and/or service provider accounts. In various embodiments, users can be or include consumers of medical information, such as individuals seeking medical information and/or advice. In various embodiments, service providers can be or include doctors, psychologists, therapists, nurses, nutritionists, and/or any other entity engaged in the practice of healthcare. In some embodiments, a user account can include user information, including health information. Health information can include medical history information, biometrics, genetics, height, weight, age, and other forms of information related to a user's health and/or useful for determining health risk, diagnosis, and/or treatment.

The system 100 can include an AI engine 20 as described herein. The AI engine 20 can be configured to process a request from a user device to interpret a context and a meaning from the request and to generate a response based on context and meaning of the request and corresponding data and information from the one or more medical information systems.

The AI engine 20 can include traditional AI functionality as well as generative AI functionality, e.g., using large language models (LLMs). The AI engine can be configured to conduct some or all of a session with a consumer user seeking medical advice, information, diagnosis, and/or treatment. In various instances, the AI engine 20 can be configured to solicit information from a user via a user device 70, collectively referred a user request. Based on the user inputs, and any other available user information, the AI engine can perform semantic and syntactic analysis of input text to determine a context and meaning of the user input and generate a response as a function thereof. The AI engine can process the request, and any related user inputs or information, to iteratively guide an interactive process responsive to the user's request. The AI engine may utilize generative AI to create new or modified content based on available medical information from one or more medical information sources, e.g., at least one library containing videos from medical experts on various topics.

The AI engine 20 can be configured to determine a language of the user and output content in the same language. In some embodiments, this may include the AI engine altering existing videos or other content to be responsive in the user's language. This may include altering the audio and visual presentation to make the response appear natural, even though the audiovisual output is modified or newly created.

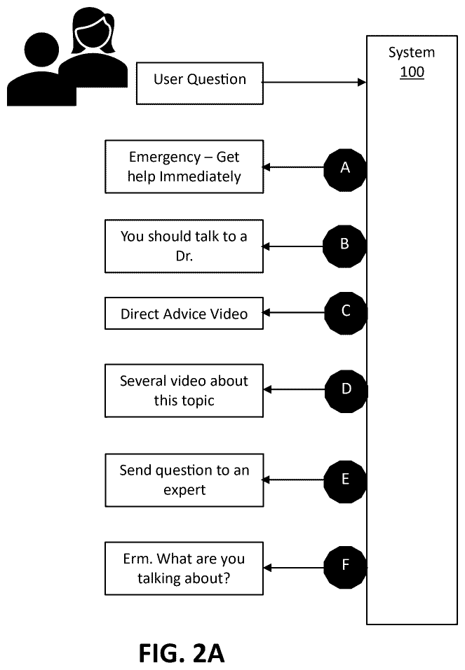

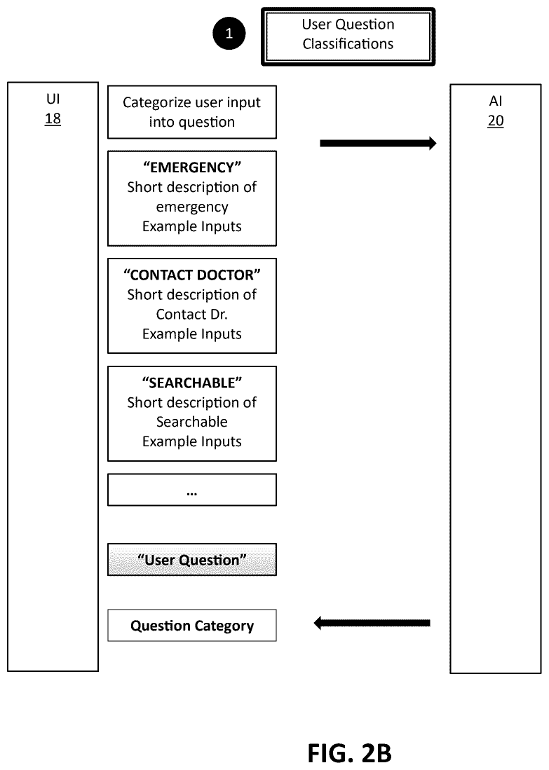

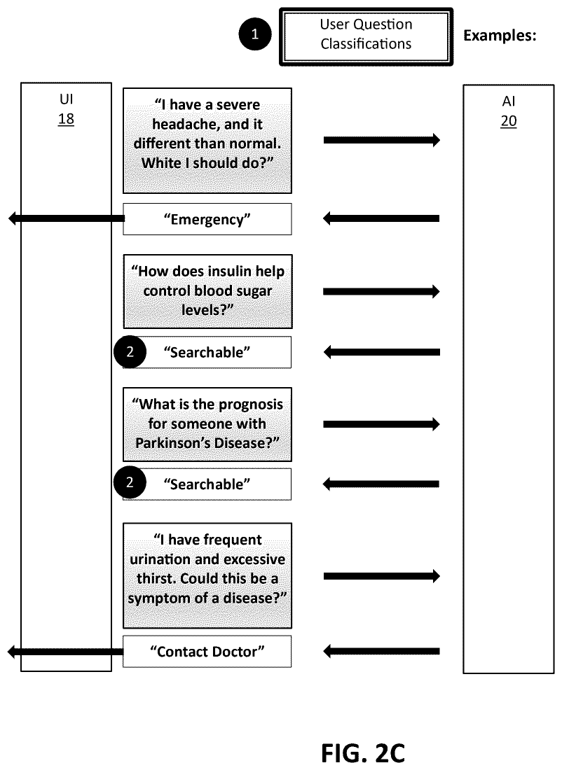

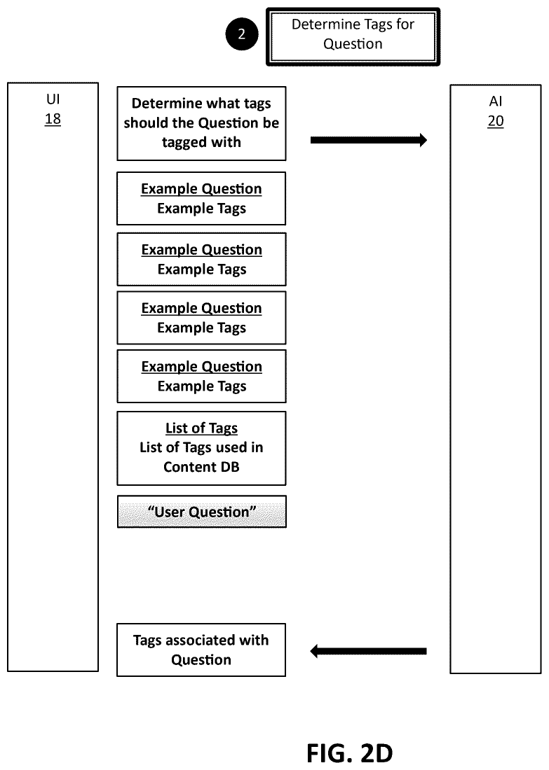

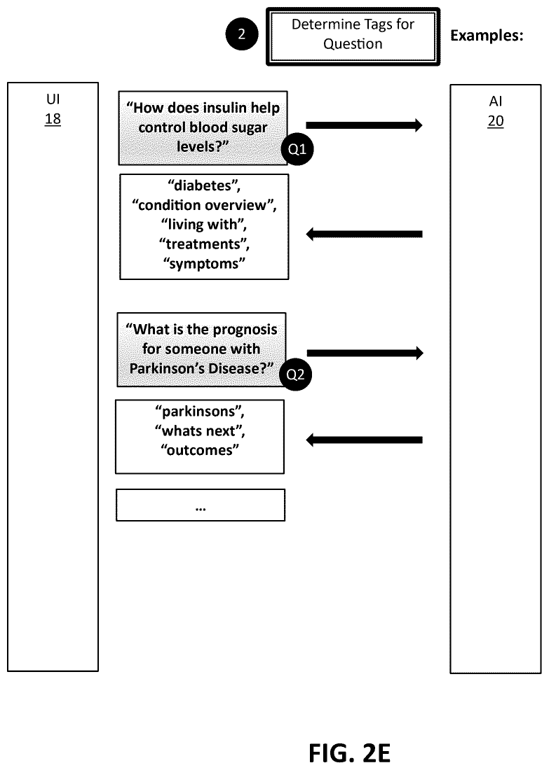

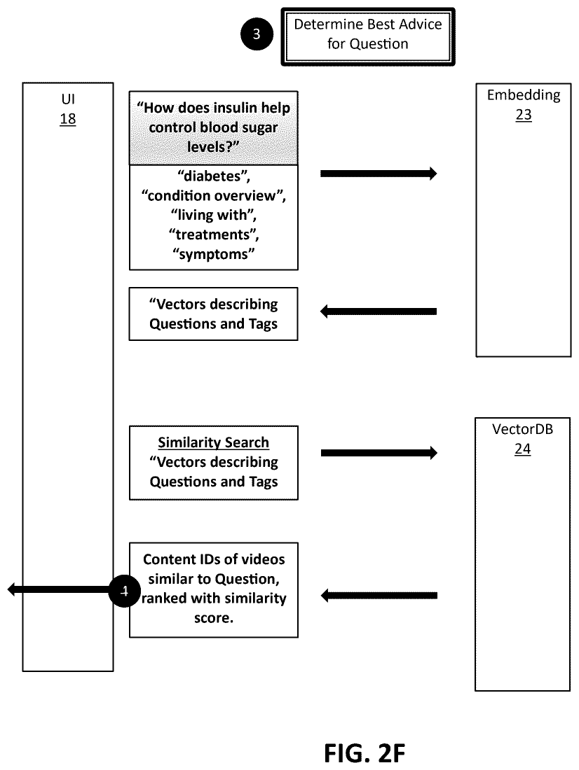

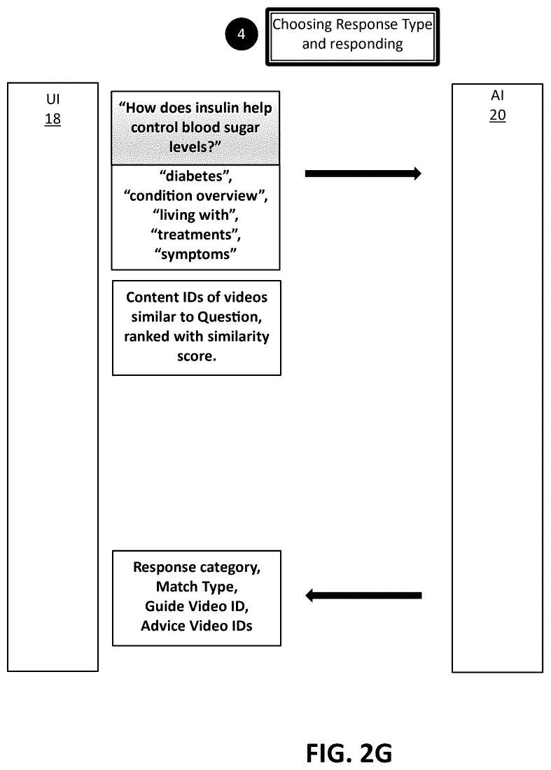

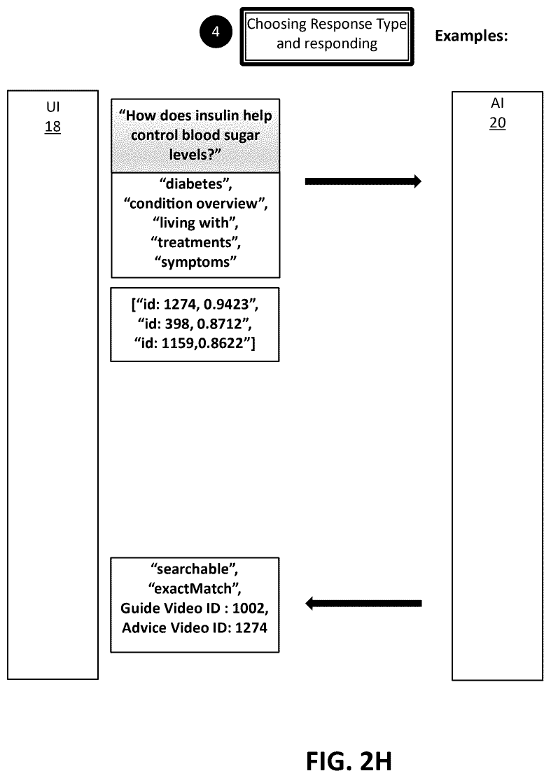

FIGS. 2A through 2H depict embodiments of flow diagrams and processing implemented by the system of FIG. 1, in accordance with aspects of the inventive concepts. For example, these flow diagram can represent how the system 100 and AI engine 20 process and interpret a user medical information request (or “Question”) received via the UI 18 and how the system 100 responds, e.g., by serving a relevant content responsive to the question, such as video, text, audio, and the like.

Referring to FIG. 2A, a flow diagram shows interactions between a user via a user device 60 and the system 100. The user, through a user device 60 and UI 18 of the system 100, provides one or more input that are received by system 100 (collectively, a “Question”) and processed, at least in part, by the AI engine 20, which can include and/or access GPT technology or other generate AI functionality. FIG. 2A shows six possible responses A-F to the user's Question. In various embodiments, these can include one or more of: A—Emergency-Get help immediately; B—You should talk to a doctor; C—present an advice video responsive to the Question; D—Present or make available several videos related to the topic of the Question; E—refer the Question to an expert, such as a doctor, and/or also initiate a live chat or video call with a doctor; and/or F—send/present a qualifying question to the user. The AI engine 20 is configured to process and interpret the Question to determine which of steps A-F should be initiated.

Referring to FIG. 2B, a first function 1 is to process classify the Question receive by UI 18 of system 100. In this embodiment, there are at least three types of responses based on the classifications: Emergency (response A in FIG. 2A), Contact Doctor (response B in FIG. 2A), and Searchable (response C, D, or E in FIG. 2A). In an Emergency situation, system 100 will instruct the user to seek immediate medical assistance. In a Contact Doctor situation, which is not interpreted as being potentially, immediately lift-threatening, the system will advise the user to schedule an appointment with a doctor to resolve the Question. In the Searchable situation, the system 100 will determine whether there is content, such as one or more video, audio, and/or text content in the data sources. If found, relevant content can be presented to the user that would be responsive to the Question.

Referring to FIG. 2C, an example of the function 1 of FIG. 2B with examples. In FIG. 2C, there are four sample questions shown based on input receive for a user. One question results in an Emergency response and another results in a Contact Doctor response. The other two questions result in a Searchable response that caused the system 100 to determine if it has access to content, such as a one or more video, it can serve in response to the question. The Searchable results are labelled “2” in FIG. 2C, as a second function.

FIG. 2D provides an embodiment of a flow for the second function 2, related to the Searchable response option. FIG. 2D is similar to FIG. 2C but with includes example of tags generated from the respective Question. For example, for the Question Q2 “How does insulin help control blood sugar levels?”, the following tags are generated “diabetes”, “condition overview”, “living with”, “treatments”, “symptoms”.

Referring to FIG. 2F, a third function 3 involves a process to determine the best advice for the user's question, utilizing the tags from the second function of FIG. 2E. In the embodiment shown, system 100 uses a database in the form of or that includes a vector database (DB). Generally, vector DB technically is known in the art, so not discussed in detail herein. Words, phrases, or entire documents, as well as images, audio, video, and other types of data, can all be vectorized. Feature vectors may be computed from raw data using machine learning methods, such as feature extraction algorithms, word embeddings or deep learning networks, where the goal is that semantically similar data items receive feature vectors close to each other. For semantic search to work effectively, embedding representations of two words having similar meaning should sufficiently capture their semantic similarity. This is where vector representations are used, and why their derivation is so important.

In FIG. 2F, the Question and tags are sent to the embedding database 23 which returns vectors describing the question and tags. A similarity search performed in the Vector DB 24, which returned content based on the vectors, such as videos from the database to relate to and/or are responsive to the Question. Embedding database 23 and vector DB 24 may be part of or otherwise accessible by the AI engine 20. In various embodiments, the content, represented by content IDs, can be ranked based on similarity scores to each content ID, e.g., by the AI engine 20. Similarities can indicate semantic similarities.

Referring to FIG. 2G, a fourth function 4 involves a process to determine a response type and to affect the response to the user via UI 18. Function 4 utilizes results from the third process 3 of FIG. 2F.

Referring to FIG. 2H, provided is an example of the function 4 of FIG. 2G with examples. In FIG. 2H, the content IDs are: “id: 1274, 0.9423”; “id: 398, 0.8712”, “id: 1159,0.8622”. The response category is Searchable (see also FIGS. 2B and 2C) and the similarity costs it “exactmatch.” The response content will include Guide Video ID: 1002 and Advice Video ID: 1274.

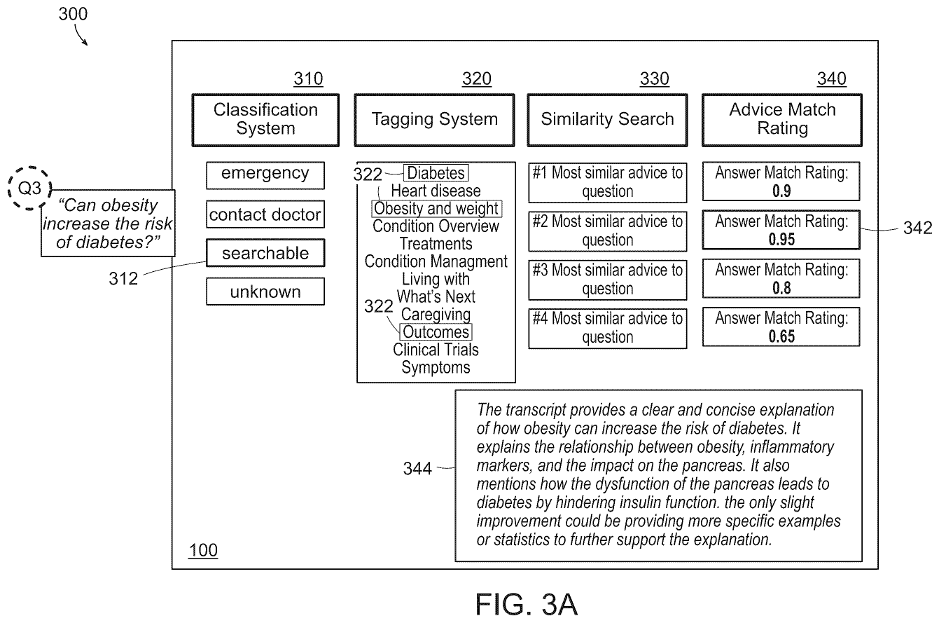

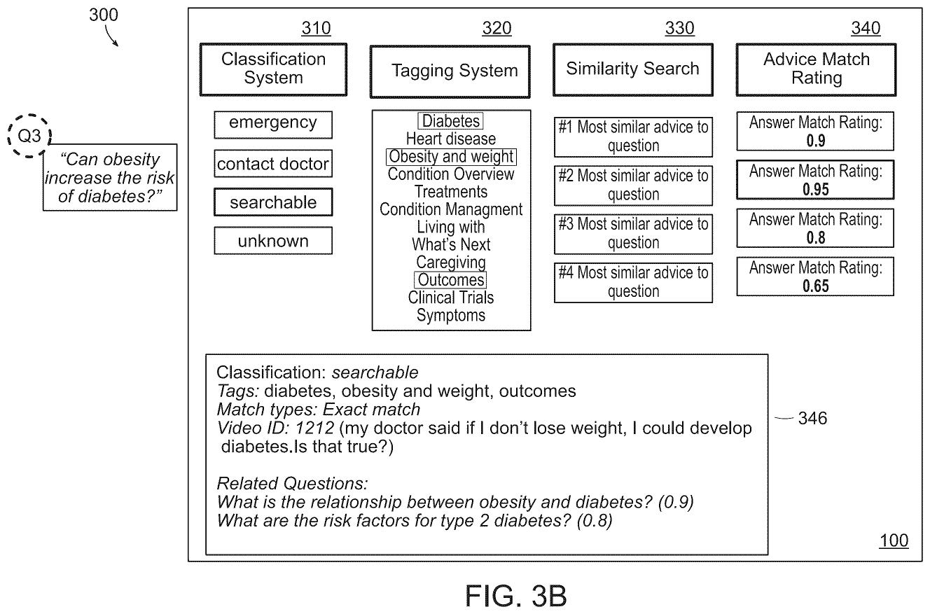

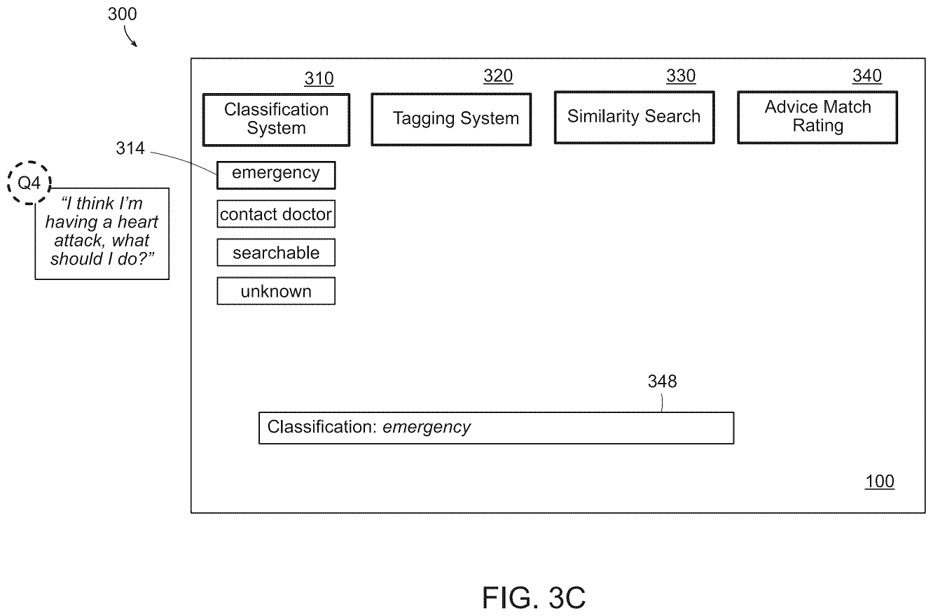

FIGS. 3A through 3C show embodiments of aspects of the system 100 of FIG. 1, which includes a classification module 310, a tagging module 320, a similarity searching module 330, and an advice match rating module 340. In this example, the question Q3 input by the user is “Can obesity increase the risk if diabetes?” The classification module 310 determines that this is a “searchable” response classification 312, e.g., rather than a medical emergency, see also FIG. 2B. Tagging module 320 interprets the question and generates search criteria or parameters in the form of the tags Diabetes, Obesity and weight, and Outcomes, 322. Similarity Search module 330 uses search parameters, e.g., the tags, to identify vectors for related content, wherein the similarity search module can be configured to determine the related content as content that is semantically similar. And Advice Match Rating module 340 assesses the content to determine a best match in view the question Q3. Answer match rating of 0.95, 342, is the highest match. An evaluation of the associated content is provided in text form 344.

FIG. 3B relates to the example of FIG. 3A, and for the same question Q3. In FIG. 3B, a summary of the processing by modules 310, 320, 330, 340 is summarized on box 346. The classification is “searchable”, the tags are “diabetes,” “obesity and weight” and “outcomes.” Four matches were found by the similarity search module 330 and there is an advice mating rating for each of the four matches. The match with the highest score, #2, is determined to be an “Exact matches.” In various embodiments, an exact match can be determined if each tag is found within the content and/or a title or question associated with the content. In various embodiments, an exact match can be determined if processing of the search criteria results in a match rating above a defined threshold. In some embodiments, the match rating threshold can be equal to or above 0.90. In some embodiments, the match rating threshold can be equal to or above 0.95. In some embodiments, the match rating threshold can be equal to or above some other value. In various embodiments, a match rating threshold can be determined semantic processing, syntactic processing, semantic processing and syntactic processing, or by some other approach for AI processing of text and/or video content in view of certain tags or other search criteria.

In FIG. 3B, the content is a video, Video ID: 1212 having an associated question of “My doctor said if I don't lose weight, I could develop diabetes. Is that true?” This question is very similar to the user's question Q3. System 100 also found a set of “Related Questions” with relatively high Advice Match Ratings: “What is the relationship between obesity and diabetes?,” with Answer Match Rating of 0.9; and “What are the risk factors for type 2 diabetes?, ” with Answer Match Rating of 0.8. There #1 and #3 from the similarity search. A question can be a Related Question if processing of the search criteria results in a match rating above a defined threshold. In some embodiments, the match rating threshold can be equal to or above 0.80. In some embodiments, the match rating threshold can be equal to or above some other value.

In various embodiments, system 100 can generate UI outputs for the user's device 60 that includes the exact match content, e.g., video ID 1212. UI is preferably user interactable. Optionally, in various embodiments the system 100 can generate UI outputs for the user's device 60 that includes one or more of the related questions and supplement content, that the user can select for viewing.

FIG. 3C is similar to the example of FIGS. 3A and 3B, in that it includes the processing of modules 310, 320, 330, 340. In the example, the classification is “emergency” 314 of the question Q4 “I think I'm having a heart attack, which should I do?”. Based on the, classification module 310 defines question as an “emergency” 314, as reflected in box 348. For an emergency classification, search of the databases for related content, such as video, is not preformed in various embodiments, because the system 100, or the AI engine 20, recognizes the urgency of the question and response with a via, via the UI of the user's device 60, to seek immediate medical attention. Therefore, tagging, similarity searching, and advice match rating need not be performed, although in some embodiments it could be to display related content.

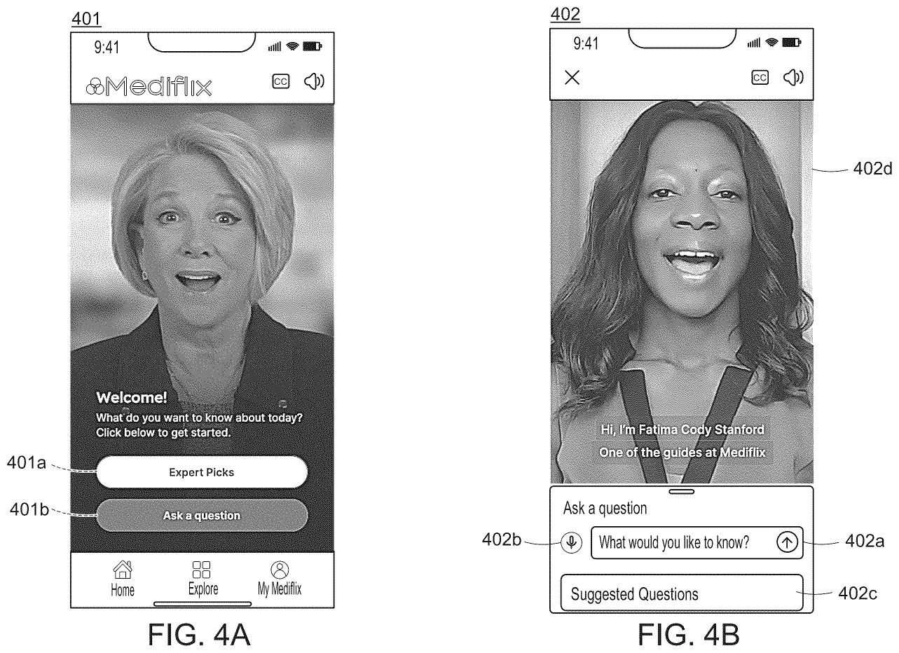

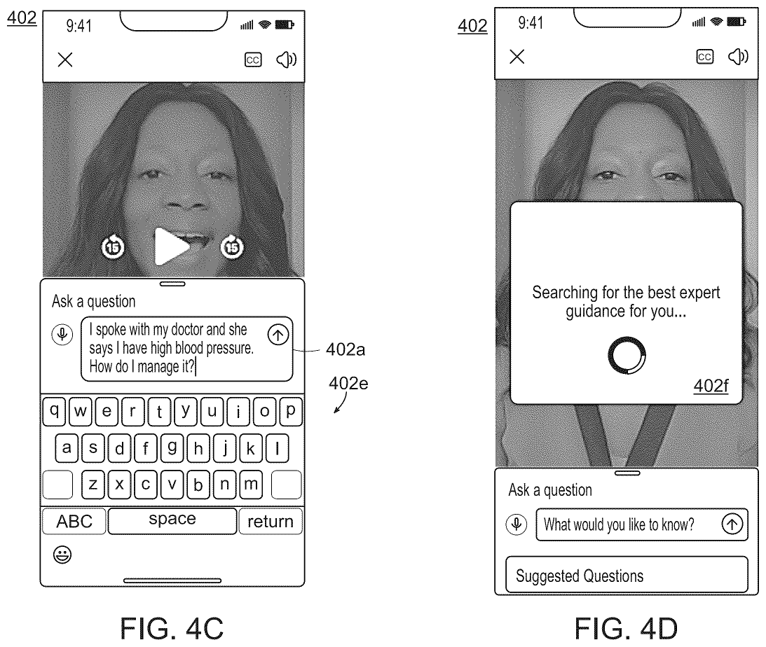

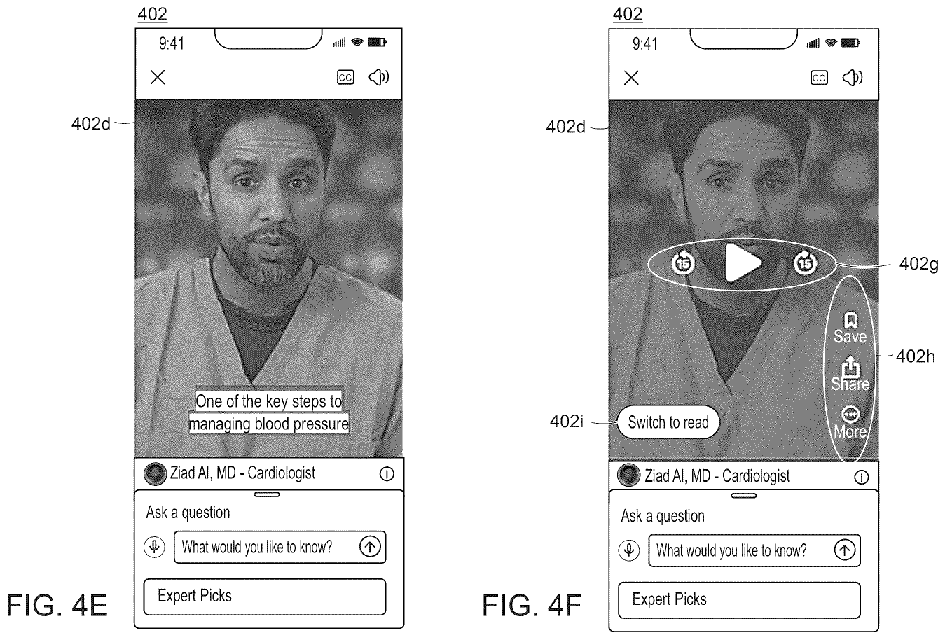

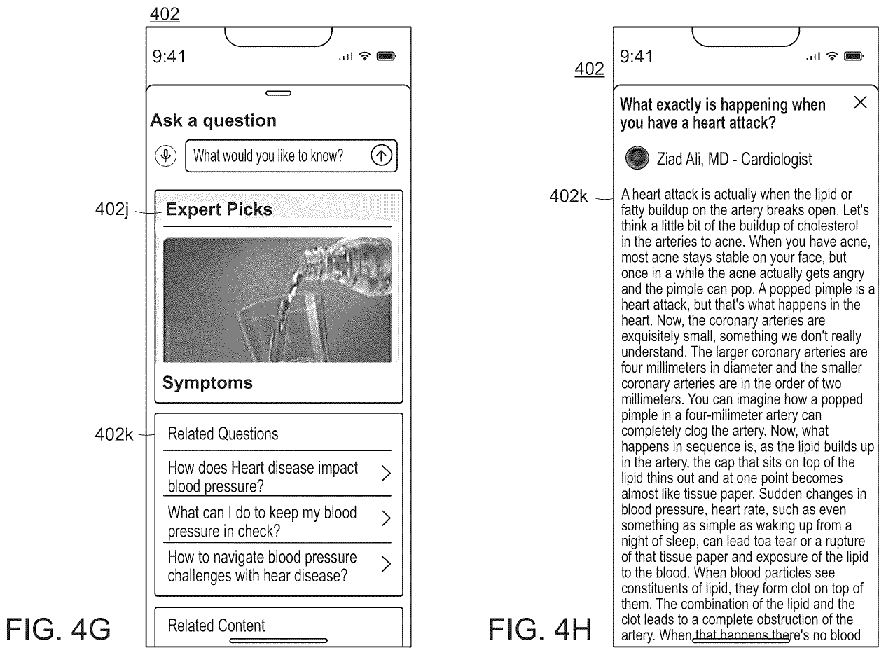

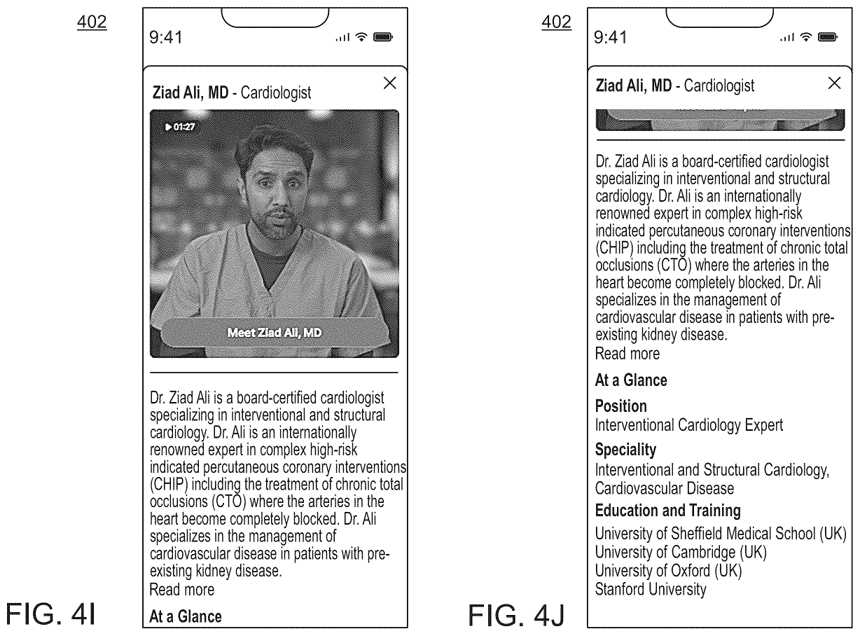

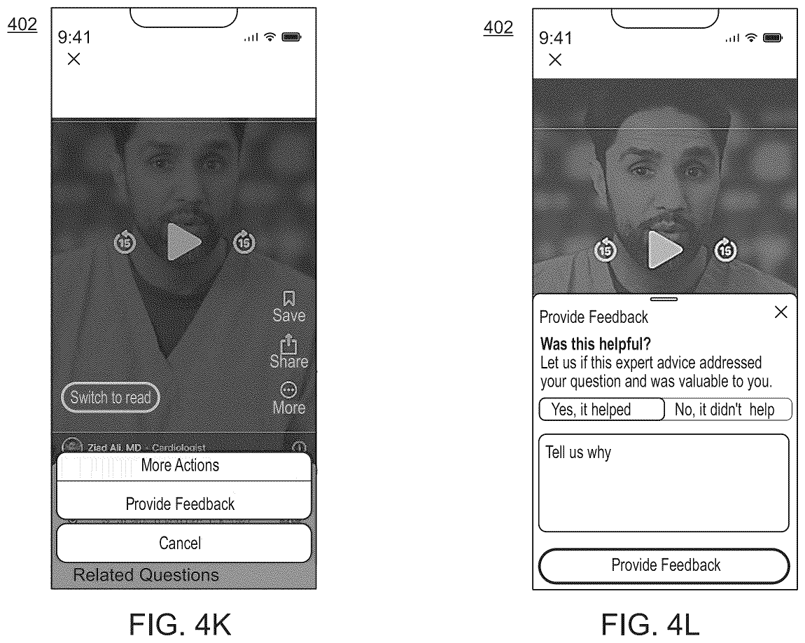

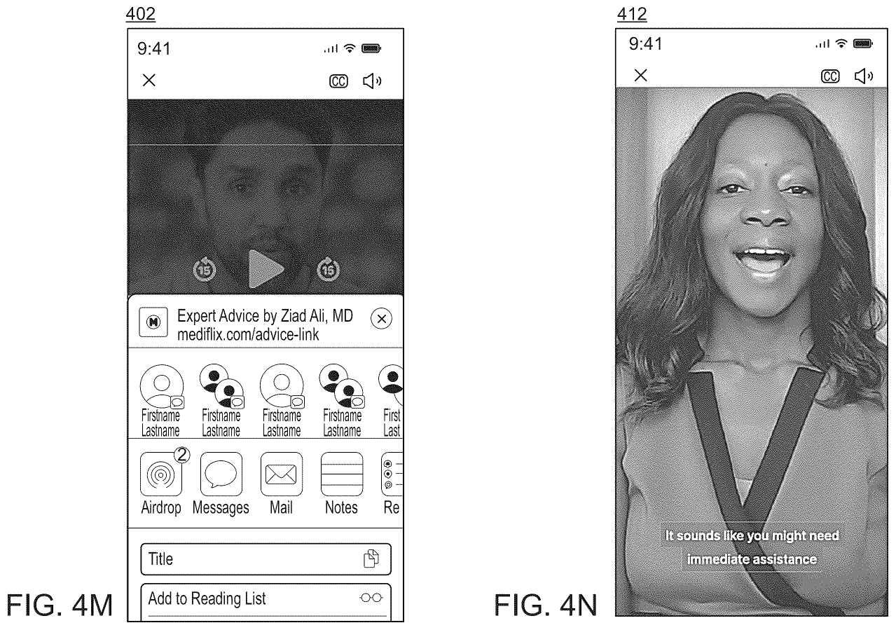

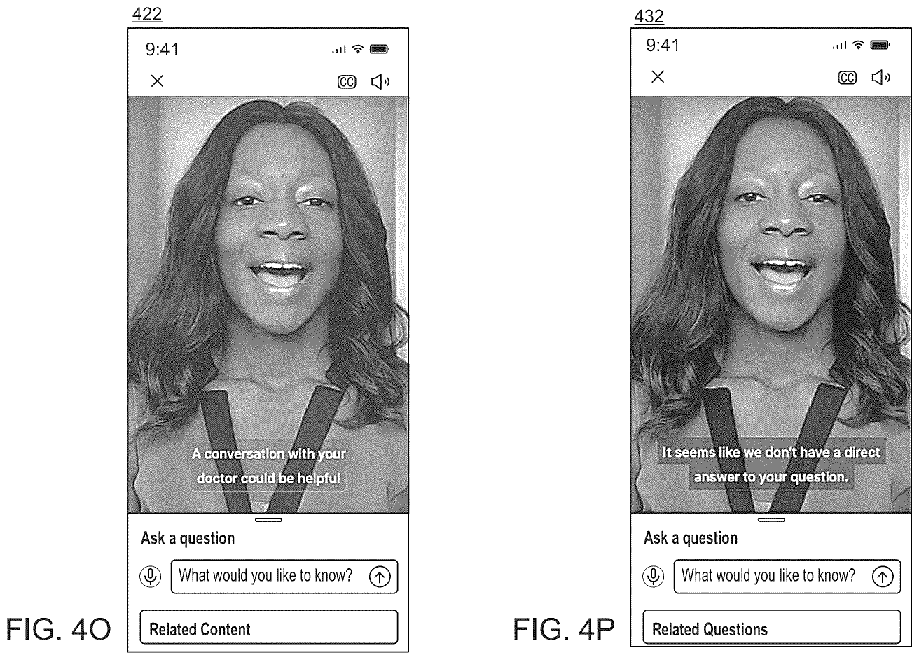

FIGS. 4A-4P depict embodiments of various screens and interactions between system 100 and user device 60 during a medical information request session. Processing by system 100, and AI engine 20, as characterized by the flow diagrams of FIGS. 2A-2H and FIG. 3A-3C is used in the rendering and presentation of the UI examples in these screens.

FIG. 4A shows a welcome screen or user interface (UI) 402 that can be generated and presented by system 100 via a user device 60. The UI can be presented in response to the initiation of an interactive medical information session between the system 100 and a user 60. UI 401 can present a welcome message that solicits a user input to direct the interaction, the welcome message could be, for example, a video welcome message. In this embodiments, UI 401 includes two user-selectable options, i.e., “Expert Picks” 401a and “Ask a question” 401b. In other embodiments, different options could be presented.

FIG. 4B depicts an embodiment of UI 402 that can be generated and presented by the system 100 via a user device 60 when the user selects the “Ask a question” 401b in UI 401 of FIG. 4A. UI 402 provides one or more user-input mechanisms that enable a user to input or initiate a medical information request, such as asking a question. In this embodiment, the user can type a question into text field 402a. UI 402 can also include an icon 402b to activate a microphone of the user device 60 allowing the user to speak its question. UI 402 can also include an output region 402d within which text and/or videos a can be presented. In FIG. 2B a welcome video is presented in region 402d to assist the user in provided an input. UI 402 can also include at least one user-selectable icon, here “Suggested Questions” 402c, that enables in the user to select from for more predefined questions, having associated searchable content. The Suggested Questions portion of UI 402 can be populated with a list of questions related to the medical information request, the user and/or user's device 60, and/or popular topics.

In FIG. 4C, the user is in the process of interacting with UI 402. The user has started typing into field 402a and the guide video in portion 402d has paused. A text keyboard 402e can be revealed with the user click-ins to the input 402a. The user request, or question, relates to high blood pressure in this example.

In FIGS. 4D, UI 402 includes a progress region 402f that indicates that system 100 is in the process of searching for content that matches with the question. In FIG. 4D, system 100 is searching for expert guidance based on the UI engine's processing of the user's medical information request, e.g., classification, tags, similarity search, and advice match rating of the user's question. The progress region 402f indicates that classification module 310, see FIGS. 3A-3C, has determined that the question “searchable.”

In FIGS. 4E and 4F, system 100 has located expert content and connected the expert content into the session within region 402d. The expert content can take the form or video, text, or some combination thereof. The expert content can be a stored video searched and retrieved based on the AI engine's processing and interpretation of the user's question as discussed above. In various embodiments, to search can consider other information about the user, e.g., medical history, preexisting conditions, and so on.

In FIG. 4E a video responsive to the user's medical information request is presented in UI region 402d, along with text output of the content of the video. In FIG. 4F the video in UI region 402d is paused. A plurality of video mechanisms 402g are provided that enable the user to rewind (15 seconds), play, and advance (15 seconds) the video. A plurality of file options mechanisms 402h are also provide, to enable to the user to save the video, share the video, or take other actions (“More”). Such “More” option can include Download, Share, Save, Copy Link, View Transcript, and so on. In this embodiment, UI portion 402d also includes a user-selectable mechanism 402i, labelled “Switch to read,” that transitions the output to read mode. The read mode can, for example, present a text version of the content of the video in region 402d.

In embodiments of FIGS. 4G and 4H, UI 402 includes a region 402j called “Expert Picks” and a region 402k called “Related Questions.” Region 402j provides links to content related to the user's question with information recommendation by at least one expert on the topic. Region 402k shows a list of questions related to the user's medical information request/question, as determined system 100 and/or AI engine 20. In FIG. 4K, UI 402 includes a region 302k that is populated content for a selection from the Experts Picks 402j from the Related Questions region 402k. Answer region 402k shows the question, in this case “What exactly is happening when you have a heart attack?” The content in region 402k answers that question, in text form in this example.

FIGS. 4I and 4J show information on the expert associated with the content presented, here Ziad Ali, MD. FIGS. 4K and 4L show screen enabling the user to provide feedback on the expert and/or the content presented. FIG. 4M shows a screen enabling a user to share the content, e.g., but choosing the “Share’ in 402h of FIG. 4F.

FIGS. 4N-4P shows guide screens presented with the classification module 310 with does not determine the question to be searchable. FIG. 4N shows an embodiment of a screen 412 when the system 100, or classification module 310, determines the entered question to indicate an emergency. FIG. 40 shows an embodiment of a screen 422 when the system 100, or classification module 310, determines the entered question indicates that the user should consider her physician. FIG. 4P shows an embodiment of a screen 432 when the system 100, or classification module 310, determines the system cannot content responsive to the entered question.

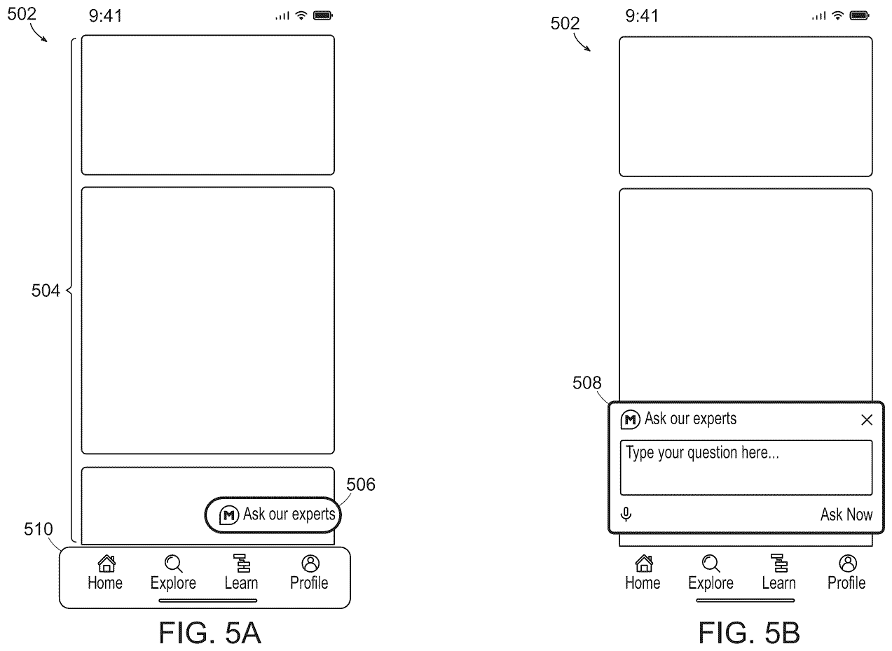

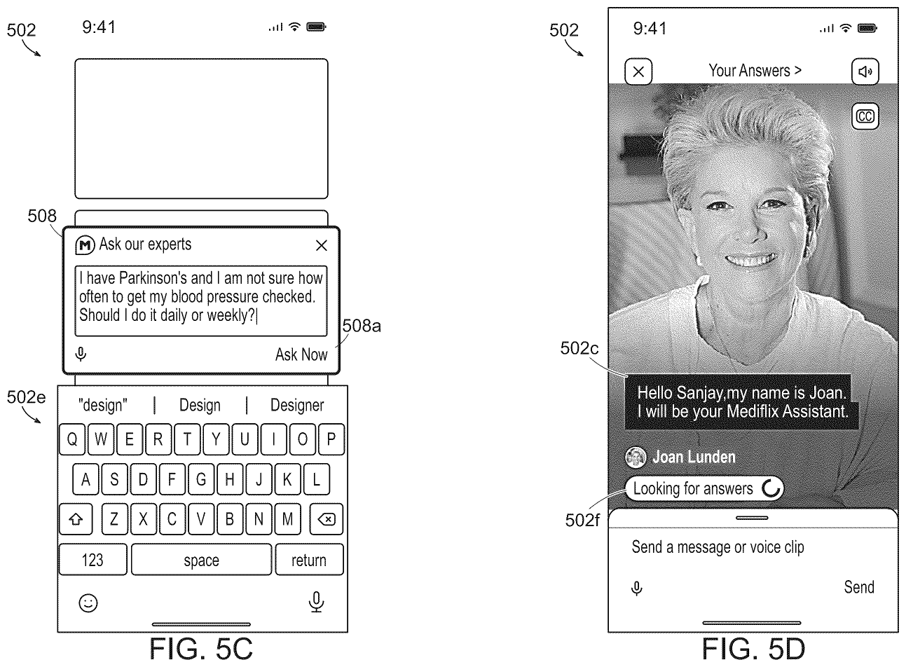

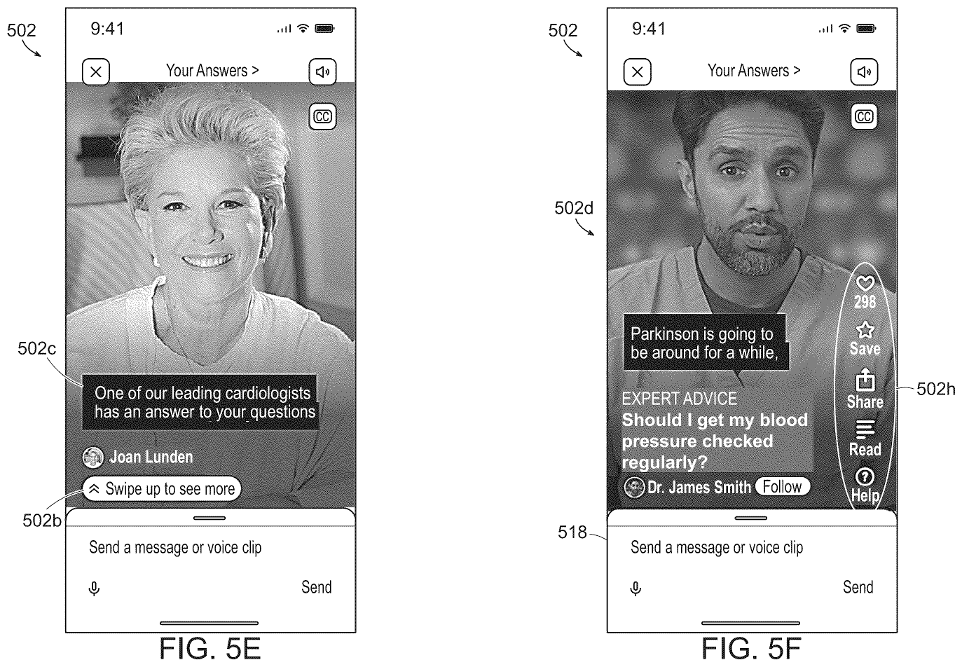

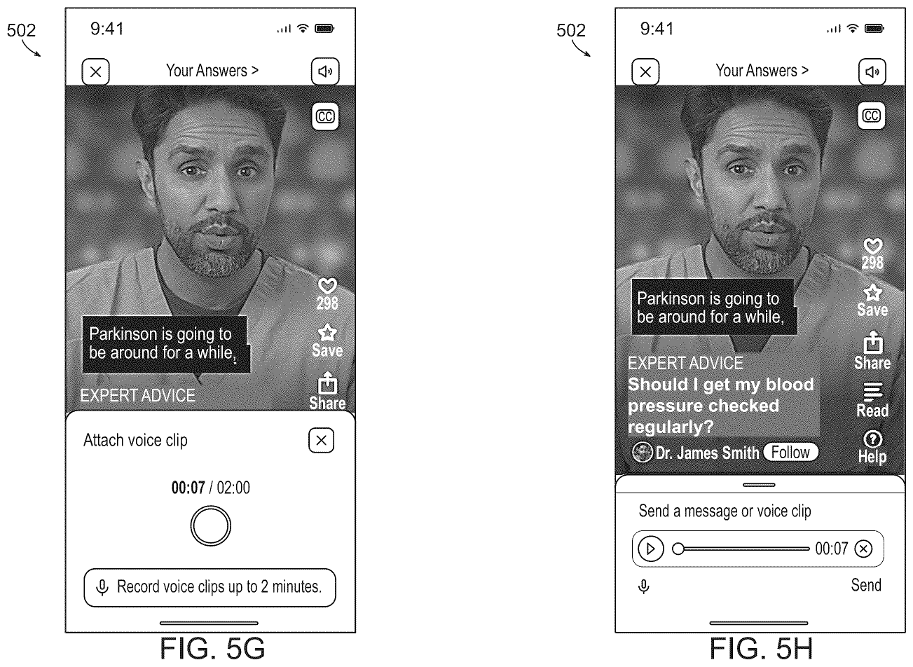

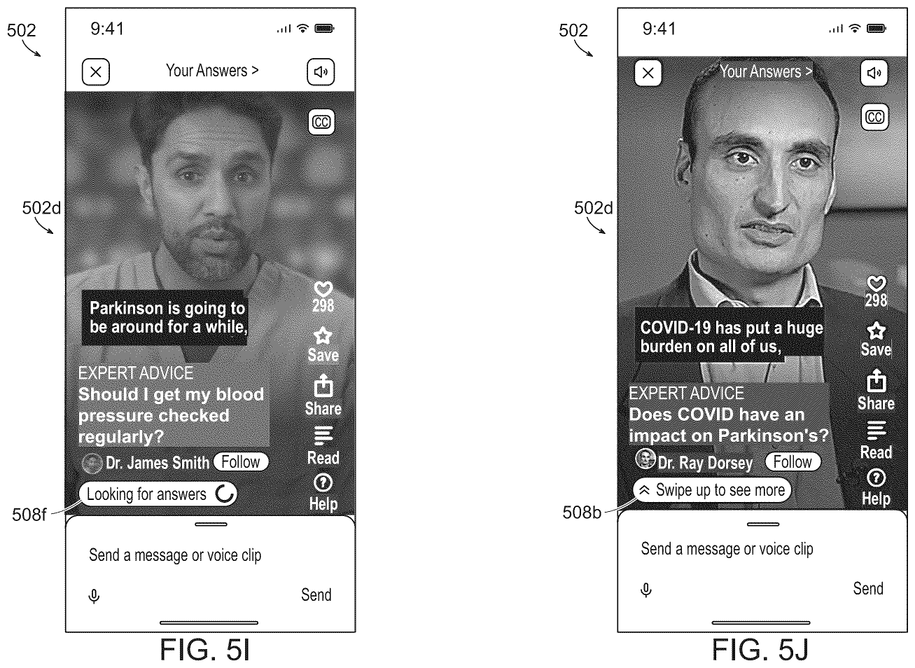

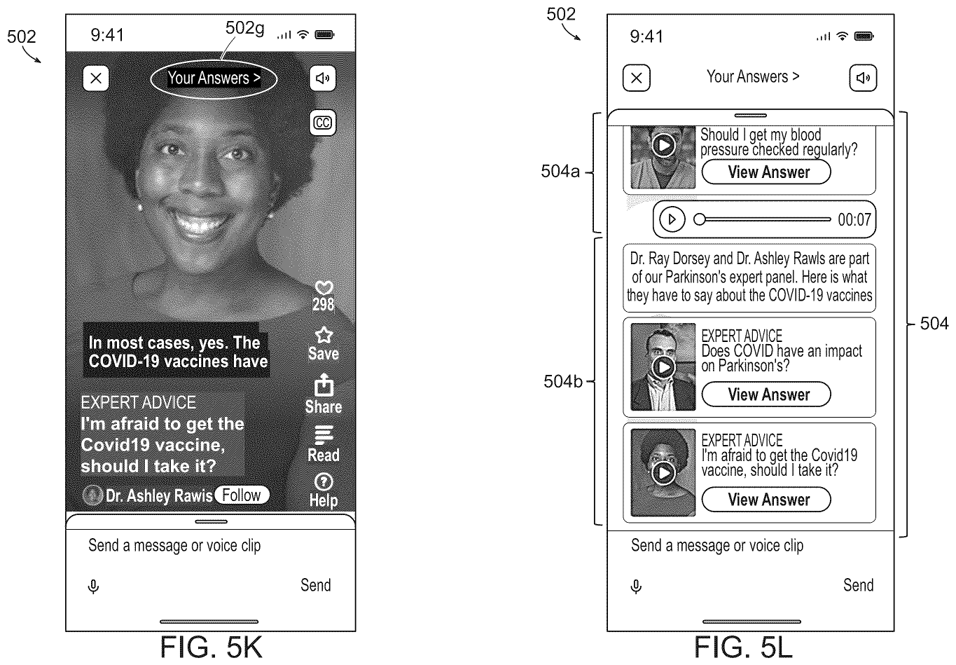

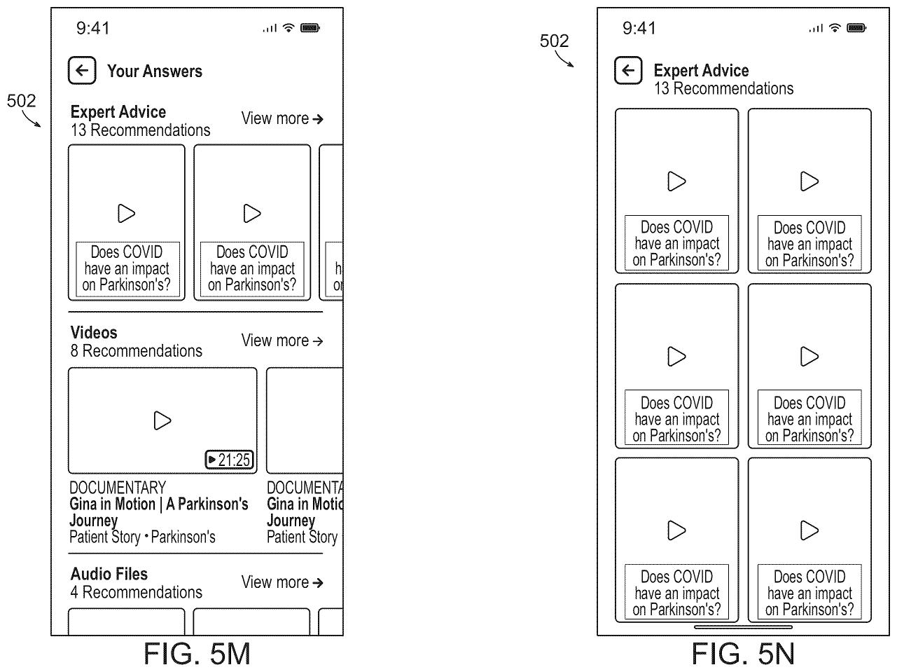

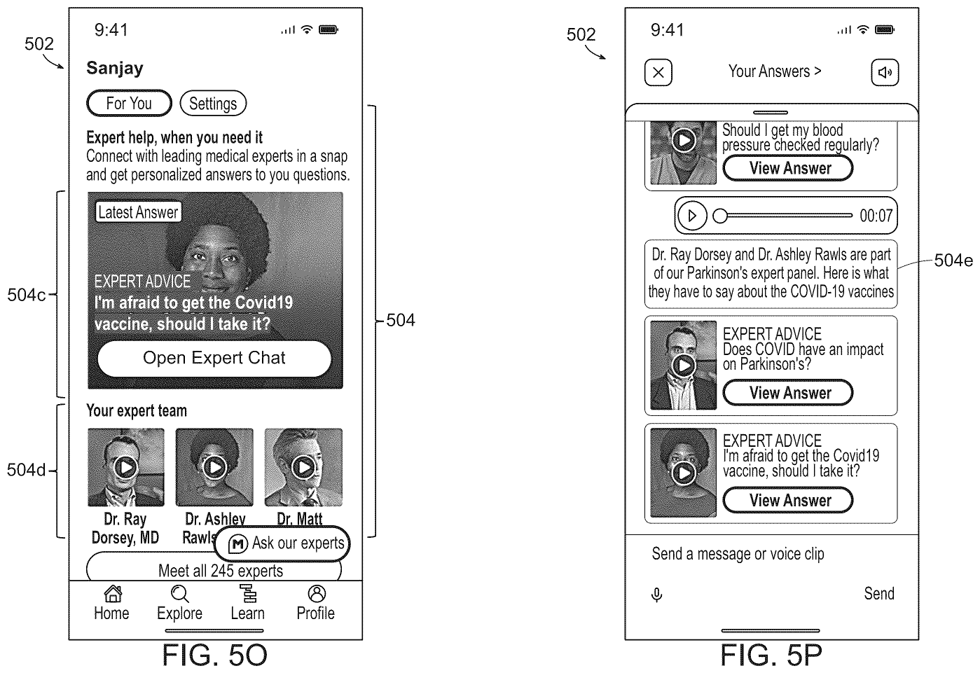

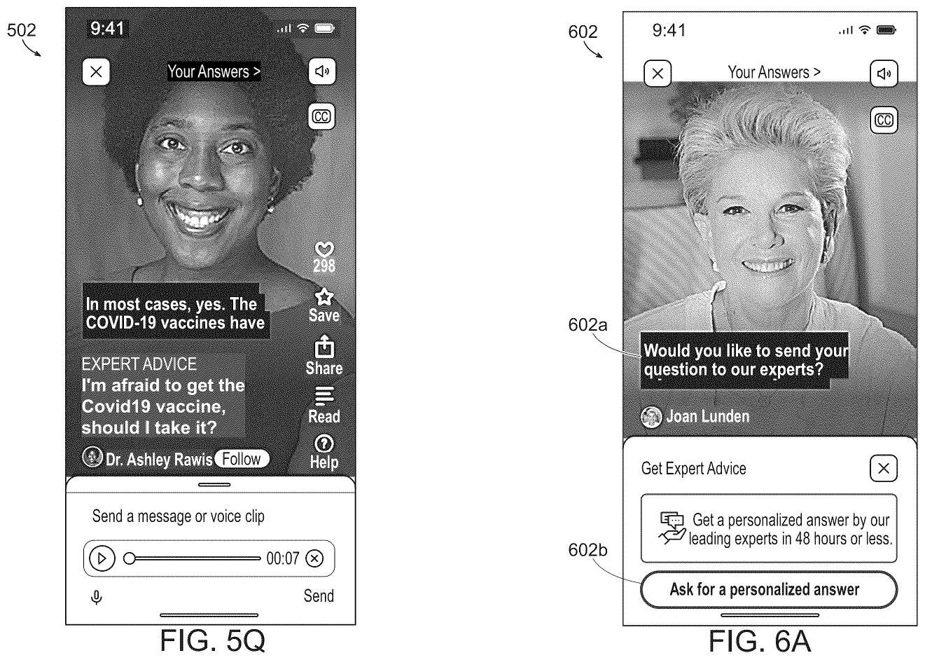

FIGS. 5A-5Q depict embodiments of various screens and interactions between system 100 and user device 60 during a medical information request session. Processing by system 100, and AI engine 20, as characterized by the flow diagrams of FIGS. 2A-2H and FIG. 3A-3C is used in the rendering and presentation of the UI examples in these screens.

Referring to FIG. 5A, a UI 502 is rendered in the user device 600 by system 100. UI 502 includes a set of panels 504 for the presentation of information and a set of icons 510 that present certain options to the user. The user may be login in to system 100, which can maintain a plurality of user accounts. The user account can include patient accounts and caregiver accounts, such as physicians, hospitals, and the like.

UI 502 includes a prompt 506 called “Ask our experts” that invites the user to ask a medical question. Upon selection prompt 506, a dialog box 508 is opened up for the user to either type in or speak in a question. As is shown in FIG. 5C, a text keyboard 502e can be revealed with the user click-ins to the input 508. The user request, or question, relates to Parkinson's Disease and high blood pressure in this example.

When the user is ready to submit the question, she selects the “Ask Now” option 508a. FIG. 5D shows a welcome screen presenting an assistant. In FIGS. 5D, UI 502 includes a a message region 502c and a progress region 502f that indicates that system 100 is in the process of searching for content that matches with the question. FIG. 5D the message region presents an introductory welcome message. FIG. 5D the message region 502c presents a message indicating expert content has been found that is responsive to the question. UI 502 also includes a mechanism 502b that enables the user to the content, called “Swipe up to see more.”

In FIG. 5F a content video is presented in region 502d from an expect, “Dr. James Smith.” A plurality of options mechanisms 502h are also provided to enable to the user to save the video, like the video, share the video, read the video, or request help. UI 502 can also include an output region 502d within which text and/or videos a can be presented. UI 502 also include a region 518 for the user to ask a question, by typing a message or my making a voice message or clip, see also FIG. 5G where a voice clip is being recorded. In FIG. 5H, the question is “Should I get my blood pressure checked regularly?” In FIG. 5I, a progress indicator 508f indicator that system 100 is searching for an answer.

In FIG. 5J a different question is asked, “Does COVID have an impact on Parkinson's.” Content from a different expert, Dr. Ray Dorsey, is presented in region 502d for this question. UI 502 also includes mechanism 502b that enables the user to the content, called “Swipe up to see more.” By selecting mechanism 502b, region 502d transitions to the content in FIG. 5K, where Dr. Ashley Rawls is presented as another expert having relevant advice for the question.

As shown in FIG. 5K, UI 502 can also include a mechanism 502g that is selectable by the user to transition of a set of content found for the user's question. FIG. 5L shows an embodiment of content of the UI 502 is the form of a region 504 comprising a set of panels showing content from different experts. In FIG. 5L, a subregion 504a is related to the first user question and subregion 504b relates to the second user question. FIGS. 5M and 5N show different embodiments of contents found based on searching of the user's questions arranged in different formats, e.g., rows or grid.

FIG. 5O, the user has asked another question: “I'm afraid to get the Covid19 vaccine, should I take it?” Region 504 of UI 502 can include two subregions, where each subregion that include different take on expert content. Subregion 504 shows content from one expert, labels “Latest Answer.” This subregion 504c has a prominent size and position within UI 502. Therefore, this subregion can be referred to as a main subregion or main panel. In other embodiments subregion 504c can be labelled differently, such as “Best Advice” or “Preferred Expert,” and the content presented could be populated accordingly the label. Either of these labels can be determined using the Advice Match Rating discussed above, e.g., see FIGS. 3A-3C. Another subregion 504d is title “Your expert team” and includes individual, selectable content tiles that when selected launch content related to the user's question and from the expert shown. Therefore, a tile can be user-selectable content.

In FIG. 5P, UI 502 also include set of a subregions with content from different experts. UI 502 also includes additional information 502e letting the user know that has least two of the experts represented share a common expertise, e.g., related to Parkinson's.

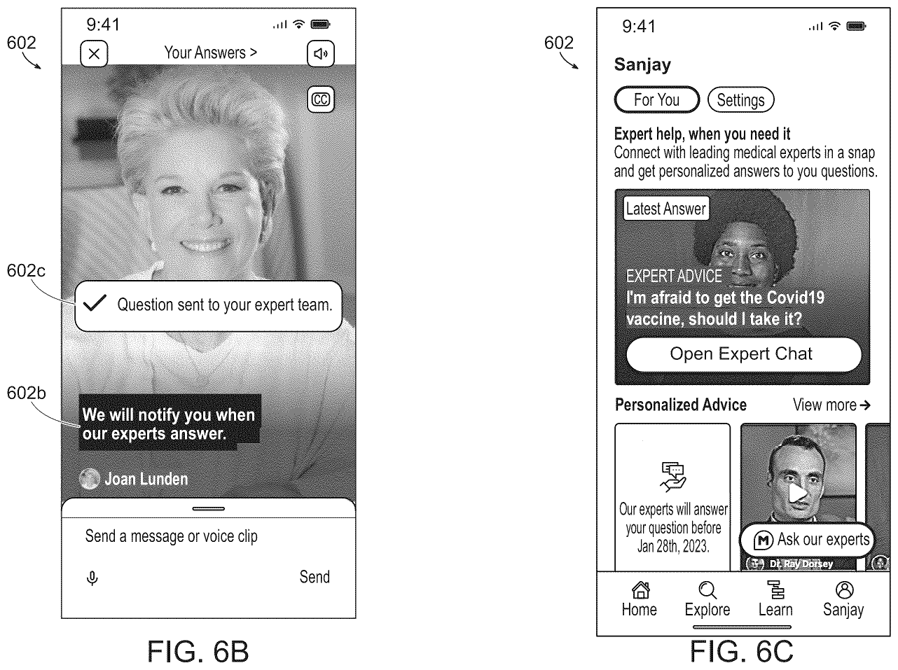

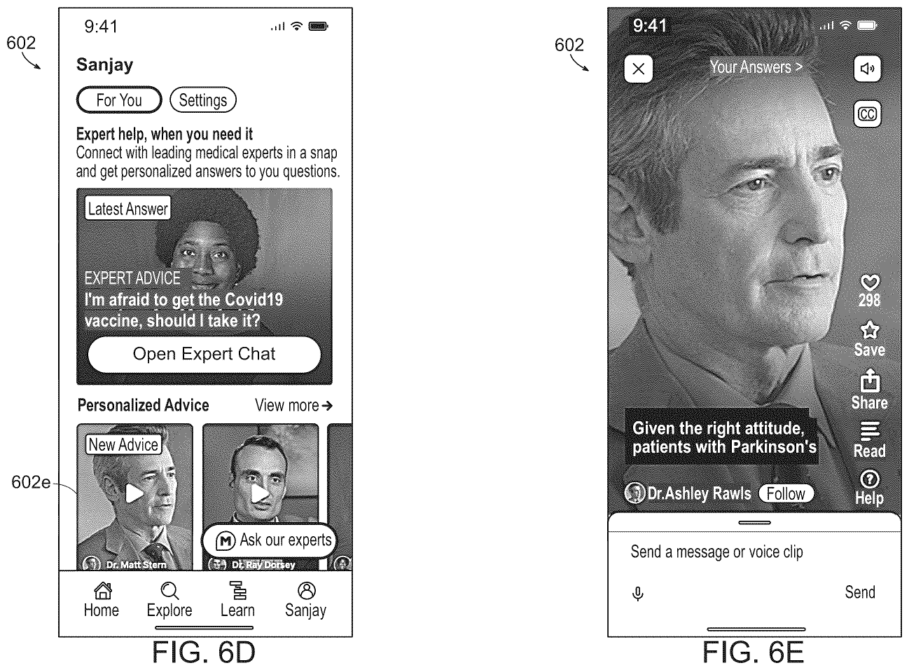

FIGS. 6A through 6E show another embodiment of screenshots resulting for a user's interaction with the system 100 of FIG. 1 and user device 60 during a medical information request session, in accordance with aspects of the inventive concepts. Processing by system 100, and AI engine 20, as characterized by the flow diagrams of FIGS. 2A-2H and FIG. 3A-3C is used in the rendering and presentation of the UI examples in these screens.

FIG. 6A shows a UI 602 presenting a welcome screen. UI 602 invites the user to input a question 602a and get medical advice, and also asked of the user wants a “personalized” response 602. FIG. 6B shows UI 602 with an indication 602c that the user's question has been sent to the “expert team,” which means system 100 and AI engine 20 are processing the questions as discussed above. A field 602d also communicated that the system win notify the user when content from the experts is ready for output to the user on user device 60.

FIGS. 6C and 6D shows screen populated with content with advice from experts, determined based on the question. These screens are similar to that shown in FIG. 5O and described above. FIG. 6D shows the screen and content presented with the user selected tile 602e, labeled New Advice” in FIG. 6D.

Below follows an itemized list of statements describing embodiments in accordance with the inventive concepts:
1. A medical information system, comprising:
   at least one processor coupled to at least one computer storage device;
   a network interface enabling connectivity with one or more medical information sources and with one or more user devices;
   an artificial intelligence (AI) engine configured to:
   process a question from a user device to determine a context and a meaning from the question; and
   generate search criteria from the context and the meaning and to search the one or more medical information sources to find expert content responsive to the question.
2. The system of statement 1, or any other statement of combination of statements, wherein the AI engine further comprises:
a classification module configured to determine a classification of the question from the context and the meaning.
3. The system of statement 2, or any other statement of combination of statements, wherein the classification module is configured to classify the question has belonging to a group consisting of: emergency, contact doctor, searchable, and unknown.
4. The system of statement 3, or any other statement of combination of statements, wherein the AI module is configured to search the one or more medical information sources when the classification is searchable.
5. The system of statement 1, or any other statement of combination of statements, wherein the expert content includes video content.
6. The system of statement 5, or any other statement of combination of statements, wherein the expert video content is prerecorded.
7. The system of statement 1, or any other statement of combination of statements, wherein the AI engine further comprises a tagging module configured to determine tags from the question.
8. The system of statement 7, or any other statement of combination of statements, wherein the tags are determined from a set of tags that include predefined, stored tags.
9. The system of statement 7, or any other statement of combination of statements, wherein the tagging module configured to use machine learning to determine the tags.
10. The system of statement 1, or any other statement of combination of statements, wherein the search criteria include the tags and the AI module further includes: a similarity search module configured to search the one or more medical information sources to determine content related to the question based, at least in part, of the tags.
11. The system of statement 10, or any other statement of combination of statements, wherein the similarity search module is configured to rank and/or order the determine content.
12. The system of statement 10, or any other statement of combination of statements, wherein the similarity search module is configured to determine the content as content that is semantically similar.
13. The system of statement 1, or any other statement of combination of statements, wherein the AI module further includes: an advice match rating module configured to assess the determine content to determine a best match for the question.
14 The system of statement 1, or any other statement of combination of statements, wherein the one or more medical information sources includes vector databases and the search criteria includes at least one vector.
15. The system of statement 1, or any other statement of combination of statements, further comprising: a user interface module configured to format the expert content for display on a user device.
16. The system of statement 15, or any other statement of combination of statements, wherein the user interface module is further configured to format the expert content into different regions on the display.
17. The system of statement 16, or any other statement of combination of statements, wherein the different regions include panels or tile, wherein a tile can be user-selectable content.
18. The system of statement 17, or any other statement of combination of statements, wherein each panel includes one or more tiles.
19. The system of statement 17, or any other statement of combination of statements, wherein each panel includes a specific type of content, including audio or video content.
20. The system of statement 17, or any other statement of combination of statements, wherein each panel includes content related to a different question.
21. The system of statement 15, or any other statement of combination of statements, wherein the user interface module is further configured to provide mechanisms for voice input and text input.
22. The system of statement 15, or any other statement of combination of statements, wherein the user interface module is further configured to include user-selected related questions in the display, each related question related to the user question.
23. The system of statement 16, or any other statement of combination of statements, wherein the user interface module is further configured to include user-selectable content from a plurality of different experts, including content related to the user question.
24. The system of statement 16, or any other statement of combination of statements, wherein the user interface module is further configured to include provide a chat session with the user.
25. The system of statement 24, or any other statement of combination of statements, wherein the chat session is with an expert related to the question.
26 The system of statement 15, or any other statement of combination of statements, wherein the user interface module is further configured to enable the user to share, save, and/or the content.
27. The system of statement 15, or any other statement of combination of statements, wherein the user interface module is further configured to enable the user to enter a subsequent question.
28. The system of statement 15, or any other statement of combination of statements, wherein the user interface module is further configured to provide a graphical mechanism to enable the user to request personalized advice.
29. The system of statement 1, or any other statement of combination of statements, wherein the AI engine includes generative AI functionality configured to generate at least some of the expert content.
30. The system of statement 29, or any other statement of combination of statements, wherein the AI engine is configured to determine a language of the user and output the expert content in the language of the user.
31 A computer-based method of providing medical information, comprising:
   providing at least one processor coupled to at least one computer storage device;
   providing a network interface enabling connectivity with one or more medical information sources and with one or more user devices;
   executing a computer program product comprising an artificial intelligence (AI) engine, including:
   processing a question from a user device to determine a context and a meaning from the question; and
   generating search criteria from the context and the meaning and searching the one or more medical information sources to find expert content responsive to the question.
32. The method of statement 29, or any other statement of combination of statements, further comprising determining a classification of the question from the context and the meaning.
33 The method of statement 31, or any other statement of combination of statements, further comprising classifying the question has belonging to a group consisting of: emergency, contact doctor, searchable, and unknown.
34 The method of statement 33, or any other statement of combination of statements, further comprising searching the one or more medical information sources when the classification is searchable.
35. The method of statement 31, or any other statement of combination of statements, wherein the expert content includes video content.
36. The method of statement 35, or any other statement of combination of statements, wherein the expert video content is prerecorded.
37. The method of statement 31, or any other statement of combination of statements, further comprising determining tags from the question.
38. The method of statement 37, or any other statement of combination of statements, further comprising determining the tags from a set of tags that include predefined, stored tags.
39. The method of statement 37, or any other statement of combination of statements, further comprising using machine learning to determine the tags.
40. The method of statement 31, or any other statement of combination of statements, wherein the search criteria include the tags and the method further comprises searching the one or more medical information sources to determine content related to the question based, at least in part, of the tags.
41. The method of statement 40, or any other statement of combination of statements, further comprising ranking and/or ordering the determine content.
42. The method of statement 40, or any other statement of combination of statements, further comprising determining the content as content that is semantically similar.
43. The method of statement 31, or any other statement of combination of statements, further comprising assessing the determined content to determine a best match for the question.
44 The method of statement 31, or any other statement of combination of statements, wherein the one or more medical information sources include vector databases and the search criteria includes at least one vector.
45. The method of statement 31, or any other statement of combination of statements, or any other statement of combination of statements, further comprising formatting the expert content for display on a user device.
46 The method of statement 45, or any other statement of combination of statements, further comprising formatting the expert content into different regions on the display.
47. The method of statement 46, or any other statement of combination of statements, wherein the different regions include panels or tiles, wherein a tile can be user-selectable content.
48. The method of statement 47, or any other statement of combination of statements, wherein each panel includes one or more tiles.
49. The method of statement 47, or any other statement of combination of statements, wherein each panel includes a specific type of content, including audio or video content.
50 The method of statement 47, or any other statement of combination of statements, wherein each panel includes content related to a different question.
51. The method of statement 45, or any other statement of combination of statements, further comprising providing mechanisms for voice input and text input.
52. The method of statement 45, or any other statement of combination of statements, further comprising including user-selected related questions in the display, each related question related to the user question.
53. The method of statement 46, or any other statement of combination of statements, further comprising including user-selectable content from a plurality of different experts, including content related to the user question.
54 The method of statement 46, or any other statement of combination of statements, further comprising including providing a chat session with the user.
55. The method of statement 53, or any other statement of combination of statements, wherein the chat is with an expert related to the question.
56. The method of statement 45, or any other statement of combination of statements, further comprising enabling the user to share, save, and/or the content.
57. The method of statement 45, or any other statement of combination of statements, further comprising enabling the user to enter a subsequent question.
58. The method of statement 45, or any other statement of combination of statements, further comprising providing a graphical mechanism to enable the user to request personalized advice.
59 The method of statement 30, or any other statement of combination of statements, wherein the AI engine includes generative AI functionality.
60. The method of statement 59, or any other statement of combination of statements, further comprising the AI engine generating at least some of the expert content.
61. The method of statement 59, or any other statement of combination of statements, further comprising the AI engine determining a language of the user and outputting the expert content in the language of the user.
62. An expert information system, comprising:
   at least one processor coupled to at least one computer storage device;
   a network interface enabling connectivity with one or more expert information sources and with one or more user devices;
   an artificial intelligence (AI) engine configured to:
   process a question from a user device to determine a context and a meaning from the question; and
   generate search criteria from the context and the meaning and to search the one or more expert information sources to find expert content responsive to the question.
63. A computer-based method of providing expert information, comprising:
   providing at least one processor coupled to at least one computer storage device;
   providing a network interface enabling connectivity with one or more expert information sources and with one or more user devices;
   executing a computer program product comprising an artificial intelligence (AI) engine, including:
   processing a question from a user device to determine a context and a meaning from the question; and
   generating search criteria from the context and the meaning and searching the one or more expert information sources to find expert content responsive to the question.

While the foregoing has described what are considered to be the best mode and/or other preferred embodiments, it is understood that various modifications can be made therein and that the invention or inventions may be implemented in various forms and embodiments, and that they may be applied in numerous applications, only some of which have been described herein. It is intended by the following claims to claim that which is literally described and all equivalents thereto, including all modifications and variations that fall within the scope of each claim.

It is appreciated that certain features of the invention, which are, for clarity, described in the context of separate embodiments, may also be provide in combination in a single embodiment. Conversely, various features of the invention which are, for brevity, described in the context of a single embodiment may also be provided separately or in any suitable sub-combination.

For example, it will be appreciated that all of the features set out in any of the claims (whether independent or dependent) can be combined in any given way.

1. A medical information system, comprising:
at least one processor coupled to at least one computer storage device;
a network interface enabling connectivity with one or more medical information sources and with one or more user devices;
an artificial intelligence (AI) engine configured to:
process a question from a user device to determine a context and a meaning from the question; and
generate search criteria from the context and the meaning and to search the one or more medical information sources to find expert content responsive to the question.

2. The system of claim 1, wherein the AI engine further comprises:
a classification module configured to determine a classification of the question from the context and the meaning.

3. The system of claim 2, wherein the classification module is configured to classify the question has belonging to a group consisting of: emergency, contact doctor, searchable, and unknown.

4. The system of claim 3, wherein the AI module is configured to search the one or more medical information sources when the classification is searchable.

5. The system of claim 1, wherein the expert content includes prerecorded, copyrighted video content.

6. The system of claim 1, wherein the AI engine further comprises:
a tagging module configured to determine tags from the question.

7. The system of claim 6, wherein the tagging module configured to use machine learning to determine the tags.

8. The system of claim 1, wherein the search criteria include the tags and the AI module further includes:
a similarity search module configured to search the one or more medical information sources to determine content related to the question based, at least in part, of the tags.

9. The system of claim 8, wherein the similarity search module is configured to rank and/or order the determined content as content that is semantically similar.

10. The system of claim 1, wherein the AI module further includes:
an advice match rating module configured to assess the determined content to determine a best match for the question.

11. The system of claim 1, wherein the one or more medical information sources includes vector databases and the search criteria includes at least one vector.

12. The system of claim 1, further comprising:
a user interface module configured to format the expert content for display on a user device.

13. The system of claim 12, wherein the user interface module is further configured to format the expert content into different regions on the display, wherein the different regions include panels or tile, wherein a tile can be user-selectable content.

14. The system of claim 13, wherein each panel includes a specific type of content, including audio or video content.

15. The system of claim 13, wherein each panel includes content related to a different question.

16. The system of claim 12, wherein the user interface module is further configured to provide mechanisms for voice input and text input.

17. The system of claim 12, wherein the user interface module is further configured to include user-selected related questions in the display, each related question related to the user question.

18. The system of claim 12, wherein the user interface module is further configured to include user-selectable content from a plurality of different experts, including content related to the user question.

19. The system of claim 12, wherein the user interface module is further configured to include provide a chat session between the user and an expert related to the question.

20. The system of claim 12, wherein the user interface module is further configured to provide mechanism that enable the user to share, save, and/or like the expert content.

21. The system of claim 12, wherein the user interface module is further configured to enable the user to enter a subsequent question and the system is configured determine and present expert content for the subsequent question.

22. The system of claim 12, wherein the user interface module is further configured to provide a graphical mechanism to enable the user to request personalized advice.

23. The system of claim 1, wherein the AI engine includes generative AI functionality.

24. The system of claim 23, wherein the AI engine is configured to determine a language of the user and output the expert content in the language of the user.

25. A computer-based method of providing medical information, comprising:
providing at least one processor coupled to at least one computer storage device;
providing a network interface enabling connectivity with one or more medical information sources and with one or more user devices;
executing a computer program product comprising an artificial intelligence (AI) engine, including:
processing a question from a user device to determine a context and a meaning from the question; and
generating search criteria from the context and the meaning and searching the one or more medical information sources to find expert content responsive to the question.
